(19) United States
(12) Patent Application Publication
(10) Pub. No.: US 2003/0008791 A1
(43) Pub. Date: Jan. 9, 2003

(54) Non-alcoholic hand sanitizer

(75) Inventor: Michael Yao-Chi Chiang, Flemington, NJ (US)

Correspondence Address: DARBY & DARBY P.C., P. O. BOX 5257, NEW YORK, NY, 10150-5257, US

(73) Assignee: Lonza Inc.

(21) Appl. No.: 10/032,204

(22) Filed: Dec. 21, 2001

Related U.S. Application Data
(60) Provisional application No. 60/296,510, filed on Jun. 6, 2001.

(51) Int. Cl. A61K 7/50

(52) U.S. Cl. 510/130; 510/138; 510/156; 510/382; 510/387; 510/504

(57) ABSTRACT

The present invention is directed to a sanitizing composition including (a) a sanitizing effective amount of an antimicrobial agent, (b) a water-soluble silicone, (c) a humectant selected from the group consisting of hydrogenated starch hydrolysates, glycerin, and mixtures thereof, and (d) water. This sanitizing composition is preferably substantially free of alcohol and more preferably contains less than about 0.5 or 1% by weight of alcohol, based upon 100% by weight of total composition. The hand sanitizer of the present invention is non-flammable, disinfects and/or sanitizes surfaces rapidly, and quickly dries after application. Another embodiment is a method of disinfecting and/or sanitizing a surface which comprises applying the aforementioned hand sanitizer composition to the surface. In particular, the hand sanitizer composition may be topically applied to hands, face and other surfaces of the body to disinfect and sanitize the same.

This application claims priority from U.S. Provisional Application Ser. No. 60/296,510 filed Jun. 6, 2001, which is hereby incorporated herein by reference.

### BACKGROUND OF THE INVENTION

Alcoholic gel-based hand sanitizers have been successfully commercialized to the health care and consumer markets. These products are convenient to use and effective at killing germs. However, since these products typically contain 60-70% ethanol, they are highly flammable. Therefore, special storage and handling procedures have been developed to store and transport these products. Not only have the flammability of these products increased their storage and distribution costs, but they have foreclosed several markets, such as the airline industry.

In addition, alcohol-based sanitizers may cause skin drying and irritation.

There is a need for a non-flammable hand sanitizer which is convenient to use, mild to the skin, and effective at killing germs.

The inventor has discovered a composition which when applied to the palms of the hands and upon rubbing, quickly dries and sanitizes the hands without the use of alcohol.

### SUMMARY OF THE INVENTION

The present invention is directed to a composition including (a) a sanitizing effective amount of an antimicrobial agent, (b) a water-soluble silicone, (c) a humectant selected from the group consisting of hydrogenated starch hydrolysates, glycerin, and mixtures thereof, and (d) water. This composition is non-flammable, bactericidal, and quick drying.

This composition is preferably substantially free of alcohol and more preferably contains less than about 0.5 or 1% by weight of alcohol, based upon 100% by weight of total composition. The hand sanitizer of the present invention is non-flammable, disinfects and/or sanitizes surfaces rapidly, and quickly dries after application.

Another embodiment is a method of disinfecting and/or sanitizing a surface which comprises applying the aforementioned hand sanitizer composition to the surface. In particular, the hand sanitizer composition may be topically applied to hands, face and other surfaces of the body to disinfect and sanitize the same.

### DETAILED DESCRIPTION OF THE INVENTION

Suitable antimicrobial agents include, but are not limited to, quaternary ammonium compounds, phenolic based compounds, and mixtures thereof. Preferred quaternary ammonium compounds include, but are not limited to, benzethonium chloride, available as Hyamine® 1622 from Lonza Inc. of Fair Lawn, NJ; benzalkonium chloride, available as Hyamine® 3500 USP from Lonza Inc.; and mixtures thereof. Preferred phenolic based compounds include, but are not limited to, trichloro-2-hydroydiphenylether (triclosan) and para-chloro-meta-xylenol (PCMX).

The water-soluble silicone functions as a spreading agent to promote drying and lubricity to the skin. A preferred water-soluble silicone is dimethicone [dimethyl, methyl (propylpoyethylene oxide polypropylene oxide, acetate) siloxane] available as Dow Corning® 190 Surfactant from Dow Coming Corp. of Midland, Mich.

Suitable humectants (moisturizers) include, but are not limited to, those listed on pages 1661 and 1662 of the 7^{th} Edition of the International Cosmetic Ingredient Dictionary and Handbook, which is hereby incorporated by reference. Non-limiting examples of suitable humectants include acetamide MEA, agarose, ammonium lactate, arginine PCA, benzyl hyaluronate, carboxymethyl chitosan succinamide, chitosan PCA, copper PCA, corn glycerides, diglycerin, dimethyl imidazolidinone, erythritol, fructose, glucamine, glucose, glucose glutamate, glucuronic acid, glutamic acid, glycereth-7, glycereth-12, glycereth-20, glycereth-26, glycereth-31, glycerin, honey, hydrogenated honey, hydrogenated starch hydrolysate, hydrolyzed corn starch, hydrolyzed wheat starch, hydroxyethyl palmityl oxyhydroxypropyl palmitamide, hydroxyethyl sorbitol, inulin, lactamide, lactamide DEA, lactamide MEA, lactic acid, lactitol, lactose, lactulose, lysine PCA, magnesium PCA, maltitol, maltose, manganese pca, mannitol, methoxypropylgluconamide, methyl gluceth-10, methyl gluceth-20, PCA, PEG-10 propylene glycol, polyamino sugar condensate, polyglucorinic acid, polyglycerin-3, polyglycerin-4, polyglycerin-6, polyglycerin-10, potassium lactate, potassium PCA, propylene-glycol, propylene glycol citrate, saccharide hydrolysate, saccharide isomerate, sodium aspartate, sodium glucuronate, sodium hyaluronate crosspolymer, sodium lactate, sodium malate, sodium PCA, sodium polyaspartate, sorbitol, sorbityl silanediol, tea-lactate, tea-PCA, urea, xylitol, xylose, and any combination of any of the foregoing. Suitable hydrogenated starch hydrolysates include, but are not limited to, Hystar® CG available from SPI Polyols of New Castle, Del.

Other useful compounds that enhance the “feel” of the sanitizer are polyglyceryl esters (PGEs). The polyglyceryl esters which may be used in the subject invention may be represented by the following general formula:

R⁵OCH₂—CH(OR⁴)—CH₂—(O—CH₂CH(OR³)—CH₂)ₙ—O—CH₂—CH(OR²)—CH₂OR¹,

wherein R is hydrogen or an acyl group (R—CO—) of a fatty acid moiety having from 8 to 22 carbon atoms, the number of R groups is from 1 to 12, and n is from 0 to 10.

The backbone is generally prepared by a condensation reaction which gives a normal distribution pattern in the final polymer. The average number of glycerin units in the base polyol will always total n+2. Generally, the PGEs used in the formulations of the invention have a molecular weight of from 600 to 3500, preferably from 800 to 1200.

The fatty acid used to prepare the PGE can be selected from a broad range of structural types such as straight chain or branched and saturated or unsaturated. In some cases, the fatty acid chain length distribution will be a specific blend or will match that found in natural oils such as, for example, almond or sunflower oils. The preferred fatty acids have from 14 to 18 carbon atoms. The 18-carbon acid is the most desirable.

The best PGEs are of decaglyceryl series where approximately 10 moles of glycerin are reacted to form the polyglycerol moiety and where there is one ester group, i.e., a monoester. In such cases, if one R equals a fatty acid moiety, then the other Rs would be hydrogen. Actually, the monoesters are not 100% but in the range of between 30% and 70% of the composition. The remainder, based on a statistical distribution, will be di-, tri- or higher esters along with some unreacted polyol. Particularly useful PGEs include decaglyceral monostearate, monoleate, decaoleate, hexaoleate, distearate, dodecaoleate, decaglyceryl dioleate, monosunflower-ate, and monoalmond-ate; triglyceryl monostearate and triglyceryl monopalmitate; and octaglyceryl dilaurate.

Naturally the solvents must be non-alcoholic. As a practical matter, water is preferred.

The composition may include other adjuvants known in the art, such as colorants, pH adjusting agents, fragrances, preservatives, thickeners, and any combination thereof. Generally, the thickener is a nonionic or a cationic compound due to the cationic nature of quaternary ammonium compounds. Non-limiting examples of preferred thickeners include hydroxypropyl methylcellulose (Methocel J12MS™ from Dow Chemical Co. of Midland, Mich.), xanthan gum (Keltrol T™ from Monsanto of Saint Louis, Mo.), acrylic copolymers (such as Structure Plus™ from National Starch and Chemical of Island Falls, Me.); and mixtures thereof.

A wide variety of preservatives can be used. These include: alkanol-substituted DMH compounds as defined in U.S. Pat. Nos. 3,987,184 and 4,172,140. These are condensation products of 5,5-dimethylhydantoin with 1, 2, or more moles of formaldehyde (e.g., 1,3-dimethylol-5,5-dimethylhydantoin, 1-methylol-5,5-dimethylhydantoin, or 3-methylol-5,5-dimethylhydantoin, 1,3-dimethyloloxymethylene-5,5-dimethylhydantoin, 1-methylol-3-methyloloxymethylene-5,5-dimethylhydantoin and 1,3-dimethyloloxymethylene-5,5-dimethylhydantoin, and mixtures thereof). A preferred preservative is Glydant® Plus Liquid, available from Lonza Inc., a combination of halogenated hydantoins and iodopropynyl butylcarbamate.

Isothiazolones may also be used. These have formula I:

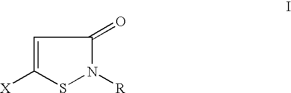

wherein X is hydrogen or halogen, preferably chlorine, and R is an alkyl chain of from 1 to 22 carbon atoms. Preferred isothiazolone components include 5-chloro-2-methyl-4-isothiazolin-3-one (CMI) and 2-methyl-4-isothiazolin-3-one (MI), and 2.8:1 mixtures of CMI/MI. Other 3-isothiazolones can be used in the invention, including 4-chloro-2-methyl-4-isothiazolin-3-one, dichloroisothiazolones such as 4,5-dichloro-2-methyl-4-isothiazolin-3-one, bromoisothiazolones such as 5-bromo-2-methyl-4-isothiazolin-3-one, n-octylisothiazolones such as 2-n-octyl-4-isothiazolin-3-one, and benzisothiazolone.

Other preservatives suitable for personal care products, such as Parabans, may also be used.

The composition is preferably substantially free of alcohol and more preferably contains less than about 0.1, 0.5, or 1% by weight of alcohol, based upon 100% weight of total composition.

To prepare the hand sanitizer of the invention, a concentrate is preferably first formed. To make the hand sanitizer, the concentrate is diluted with 5 to 20 times its weight of a solvent with or without dissolved constituents.

The concentrate will contain the antimicrobial agent, the spreading agent, and the humectant dissolved in the solvent. Optionally, the preservative, colorant and fragrance may be included.

Before use, the concentrate is diluted, preferably with the same solvent as was used in the concentrate. The diluent will contain, if desirable, a thickener, the preservative, the colorant and/or the fragrance, though the last three may also be included in the concentrate. Since these latter components are quite soluble, it is of little consequence whether they form part of the concentrate or are dissolved in the diluent. The thickener cannot be added to the concentrate.

Table I illustrates the components and the ranges of components present in the concentrate.

TABLE I

| Component | Broad | Preferred |
|---|---|---|
| A. Antimicrobial agent | 0.5-2.0% | 1-2% |
| B. Water-soluble silicone | 5-20% | 8-12% |
| C. Humectant | 30-60% | 35-45% |
| D. Preservative | 0.05-0.2% | |
| E. Colorant | up to 0.01% | |
| F. Fragrance | up to 0.5% | |
| G. Solvent | to make 100% | |

The range of compounds in the use dilution is shown in Table II. The compositions may be obtained by diluting the concentrate shown in Table I or by blending all of the components at essentially the same time.

TABLE II

| Component | Broad | Preferred |
|---|---|---|
| a. Antimicrobial agent | 0.05-0.5% | 0.1-0.2% |
| b. Water-soluble silicone | 0.1-50% | 0.5-2.0% |
| c. Humectant | 1-8% | 2-6% |
| d. Preservative | 0.05-0.2% | 0.1-0.2% |
| e. Thickener | up to 0.8% | |
| f. Colorant | up to 0.01% | |
| g. Fragrance | up to 0.5% | |
| h. Solvent | 90-99% | 92-96% |

Generally, the use dilution and concentrate are stable single phase solutions. The components selected are generally recognized as safe and effective. The use dilution has a pH of broadly from 5 to 8 and preferably from 6 to 7. The viscosity may vary from 1 to 500 cps depending on the application. Where a thickener is used, it may be necessary to adjust the pH to about 8 (using any appropriate base such as an alkali or alkaline earth metal, preferably sodium hydroxide) to form a clear solution. After the clear solution is formed, the pH of the use dilution may be adjusted to be in the preferred range without loss of clarity.

The following example illustrates the invention without limitation. All parts and percentages are given by weight unless otherwise indicated.

### EXAMPLE 1

A non-alcoholic hand sanitizer concentrate of the invention may be prepared by mixing the ingredients shown in Table III below.

TABLE III

| Ingredient | Function | Parts By Weight |
|---|---|---|
| Benzethonium chloride USP | Biocide | 2 |
| Dow Coming ® 190 Surfactant | Spreading agent | 10 |
| Hystar ® CG | Humectant | 40 |
| Water | Solvent | 48 |

| | |
|---|---|
| TOTAL | 100 |

The use dilution of the invention may be prepared by diluting 10 parts of the concentrate with 90 parts of the diluent shown in Table IV.

TABLE IV

| Ingredient | Function | Parts By Weight |
|---|---|---|
| Methocel J12MS | Thickener | 0.3 |
| Glydant ® Plus Liquid | Preservative | 0.1 |
| Fragrance | Aesthetic | 0.4 |
| Water | Solvent | ˜89 |

| | |
|---|---|
| TOTAL | 90 |

The resulting non-alcoholic hand sanitizer composition shown in Table V below was prepared by mixing the concentrate of Table III with the diluent of Table IV:

TABLE V

| Ingredient | Function | Parts By Weight |
|---|---|---|
| Benzethonium chloride USP | Antimicrobial agent | 0.2 |
| Dow Coming ® 190 Surfactant | Spreading agent | 1 |
| Hystar ® CG | Humectant | 4.0 |
| Methocel J12MS | Thickener | 0.3 |
| Glydant ® Plus Liquid | Preservative | 0.1 |
| Fragrance | Aesthetic | 0.4 |
| Water | Solvent | 93.88 |

| | |
|---|---|
| TOTAL | 100 |

The above composition is a clear liquid having a pH of about 6-8 and a specific gravity of about 8.5 lbs/gal. The sanitizer is used by applying a small amount to hands and rubbing the hands together as washing hands.

### EXAMPLE 2

The antimicrobial efficacy of the non-alcoholic hand sanitizer formulation of Example 1 was determined against the bacteria listed in Table VI as follows. The bacteria were grown overnight on separate agar-solidified growth mediums. Each growth medium was then suspended in a phosphate buffer and diluted to a concentration of 3×10⁸ bacteria per ml. 1 ml of the cell suspension was added to 10 ml of the non-alcoholic hand sanitizer formulation and mixed well. After one minute, an aliquot was removed and transferred to a neutralizing tube containing Letheen broth, modified to include additional Tween® 80, available from Uniquema Chemicals of Wilmington, Del., and lecithin. The number of bacteria in the neutralizing tube were counted by performing serial dilutions in microwell plates.

A control containing 0.003 8% (w/w) potassium phosphate buffer in water was also tested by this method.

The bacterial reduction for the non-alcoholic hand sanitizer formulation was calculated based on the results from the unchallenged control. The results are shown in Table VI.

TABLE VI

| | Percent Bacterial | Percent Bacterial |
|---|---|---|
| Sample | Reduction (Control) | Reduction (Example 1) |
| S. aureus (ATCC 6538) | 0 | 99.999 |
| S. epidermidis | 0 | 99.995 |
| (ATCC 12228) | | |
| E. coli (ATCC 11229) | 0 | 99.999 |
| E. coli 0157:H7 | 0 | 99.999 |
| (ATCC 35150) | | |
| P. aeruginosa | 0 | 99.999 |
| (ATCC 15442) | | |
| S. typhi (ATCC 6539) | 0 | 99.999 |
| L. monocytogenes | 0 | 99.999 |
| (ATCC 7644) | | |
| S. marcescens | 0 | 99.999 |
| (ATCC 14756) | | |

Table VII shows the log of the bacterial count after exposure to the control solution for 1 minute and the log of the bacterial count before exposure to the non-alcoholic hand sanitizer formulation.

TABLE VII

| | Log of Bacterial Count | Log of Bacteria Killed |
|---|---|---|
| | after Exposure to Control | After Exposure to the |
| Bacteria | for 1 Minute | Formulation of Example 3 |
| S. aureus | 5 | N/A |
| S. epidermidis | 4.5 | 4.5 |
| E. coli | 5 | 5 |
| E. coli 0157:H7 | 5 | 5 |
| P. aeruginosa | 5 | 5 |
| S. typhi | 6 | 6 |
| L. monocytogen | 4.5 | 4.5 |
| S. marcescens | 5 | 5 |

All patents, applications, articles, publications, and test methods mentioned above are hereby incorporated by reference.

Many variations of the present invention will suggest themselves to those skilled in the art in light of the above detailed description. Such obvious variations are within the full intended scope of the appended claims.

In the claims:

1. A sanitizing composition comprising:
a sanitizing effective amount of an antimicrobial agent;
a water-soluble silicone;
a humectant selected from the group consisting of hydrogenated starch hydrolysates, glycerin, and mixtures thereof; and
water,
wherein the composition is substantially free of alcohol.

2. A sanitizing composition as defined in claim 1, wherein the concentration of the antimicrobial agent ranges from about 0.05 to about 0.5 percent by weight, the concentration of the water-soluble silicone ranges from about 0.1 to about 5.0 percent by weight and the concentration of the humectant ranges from about 1 to about 8 percent by weight, based upon 100% total weight of composition.

3. A sanitizing composition as defined in claim 1, wherein the concentration of the antimicrobial agent ranges from about 0.1 to about 0.2 percent by weight, the concentration of the water-soluble silicone ranges from about 0.5 to about 2.0 percent by weight and the concentration of the humectant ranges from about 2 to about 6 percent by weight, based upon 100% total weight of composition.

4. The sanitizing composition as defined in claim 2, wherein the antimicrobial agent is a quaternary ammonium compound.

5. The sanitizing composition as defined in claim 4, wherein the quaternary ammonium compound is benzethonium chloride.

6. The sanitizing composition as defined in claim 4, wherein the quaternary ammonium compound is benzalkonium chloride.

7. The sanitizing composition as defined in claim 1, wherein the antimicrobial agent is a phenolic based compound.

8. The sanitizing composition as defined in claim 7, wherein the phenolic based compound is trichloro-2-hydroxydiphenylether.

9. The sanitizing composition as defined in claim 7, wherein the phenolic based compound is para-chloro-meta-xylenol.

10. The sanitizing composition as defined in claim 1, wherein the water-soluble silicone is dimethicone.

11. The sanitizing composition as defined in claim 1, wherein the compositition also contains a polyglycerol ester.

12. A sanitizing composition comprising:
from about 0.05 to about 0.5 percent by weight of an antimicrobial agent;
from about 0.1 to about 5.0 percent by weight of a water-soluble silicone;
from about 1 to about 8 percent by weight of a humectant selected from the group consisting of hydrogenated starch hydrolysates, glycerin, and mixtures thereof; and
water, and, optionally,
from about 0.05 to about 0.2 percent by weight of a preservative,
up to about 0.8 percent by weight of a thickener,
up to about 0.01 percent by weight of a colorant, and/or
up to about 0.5 percent by weight of a fragrance, or any combination thereof,
wherein the composition is substantially free of alcohol.

13. A sanitizing composition comprising:
a sanitizing effective amount of benzethonium chloride;
dimethicone;
a hydrogenated starch hydrolysate humectant;
an alkanol hydantoin preservative; and
water,
wherein the composition is substantially free of alcohol.

14. A sanitizing composition as defined in claim 13, wherein the concentration of the benzethonium chloride ranges from about 0.1 to about 0.2 percent by weight, the concentration of the dimethicone ranges from about 0.5 to about 2.0 percent by weight and the concentration of the hydrogenated starch hydrolysate ranges from about 2 to about 6 percent by weight, based upon 100% total weight of composition.

15. A method of disinfecting and sanitizing a surface, the method comprising applying to the surface the sanitizing composition of claim 13.

16. The method of claim 13, wherein the surface is skin of a human body.

17. A sanitizing concentrate comprising, in water:
from about 0.5 to about 2 percent by weight of an antimicrobial agent;
from about 5 to about 20 percent by weight of a water-soluble silicone; and
from about 30 to about 60 percent by weight of an humectant.

18. The sanitizing concentrate as defined in claim 17, wherein the concentration of the antimicrobial agent ranges from about 1 to about 2 percent by weight, the concentration of the water-soluble silicone ranges from about 8 to about 12 percent by weight and the concentration of the humectant ranges from about 35 to about 45 percent by weight, based upon 100% total weight of composition.

19. The sanitizing concentrate as defined in claim 18, wherein the antimicrobial agent is benzethonium chloride; the water-soluble silicone is dimethicone; and the humectant is an hydrogenated starch hydrolysate.
